(19) United States
(12) Patent Application Publication
(10) Pub. No.: US 2016/0278708 A1
(43) Pub. Date: Sep. 29, 2016

(54) BIOLOGICAL PARAMETER ESTIMATION

(71) Applicant: IMRA EUROPE S.A.S., Sophia Antipolis Cedex (FR)

(72) Inventor: Sacha Vrazic, Sophia Antipolis Cedex (FR)

(21) Appl. No.: 15/080,860

(22) Filed: Mar. 25, 2016

(30) Foreign Application Priority Data
Mar. 27, 2015 (DE) 102015104726.8

(51) Int. Cl. A61B 5/00; A61B 5/0205

(52) U.S. Cl. CPC A61B 5/6892; A61B 5/7214; A61B 5/725; A61B 5/6893; A61B 5/0205; A61B 2562/0219; A61B 2562/0247; A61B 5/024

(57) ABSTRACT

A biological parameter of a subject is estimated, which is present on a support, the support comprising at least two sensors each measuring a variation of pressure, wherein at least one accelerometer is connected to the support. A sensor-specific model is provided for each of the at least two sensors based on signals from the at least two sensors, the signals corresponding to the variation of pressure measured by the at least two sensors, respectively. In a selection process, at every time frame T, one sensor is selected out of the at least two sensors, to be used for estimating the biological parameter of the subject, based on signals from the at least one accelerometer. In an estimation process, the biological parameter of the subject is estimated using, at every time frame T, the sensor-specific model provided for the selected one sensor.

### CROSS REFERENCE TO RELATED APPLICATIONS

This application claims the benefit of German Patent Application No. 102015104726.8, filed Mar. 27, 2015, incorporated herein by reference.

### BACKGROUND OF THE INVENTION

It is sometimes desirable to make a human body physiological parameter estimation, in particular pulsation and breathing, e.g. for drivers and passengers of a vehicle. Such biological parameter monitoring has been described in the patent literature in French Patent Application Publications FR 2,943,233 A1, FR 2,943,234 A1 and FR 2,943,236 A1. For example, pulsation and breathing signals of a supported person can be acquired using piezoelectric sensors. As is known to those of skill in the art, piezoelectric sensors measure a variation of pressure, such that piezoelectric sensors embedded in a seat at home or in a car can measure a displacement created by blood flow pressure.

### SUMMARY OF THE INVENTION

Embodiments of the present invention provide improved biological parameter monitoring. More particularly, embodiments described herein provide a robust biological parameter estimation for a subject on a support, e.g. a seat or bed, in which at least two sensors for measuring variation of pressure are embedded.

According to an example embodiment of the invention, a biological parameter of a subject who is present on a support is estimated, the support comprising at least two sensors each measuring a variation of pressure, wherein at least one accelerometer is connected to the support. A sensor-specific model is provided for each of the at least two sensors based on signals from the at least two sensors, the signals corresponding to the variation of pressure measured by the at least two sensors, respectively. In a selection process, at every time frame T, one sensor is selected out of the at least two sensors, to be used for estimating the biological parameter of the subject, based on signals from the at least one accelerometer. In an estimation process, the biological parameter of the subject is estimated using, at every time frame T, the sensor-specific model provided for the selected one sensor, thereby providing a robust pulsation estimation by changing the sensor on which the estimation should be done.

In the following the invention will be described by way of embodiments thereof with reference to the accompanying drawings.

### BRIEF DESCRIPTION OF THE DRAWINGS

FIG. 1 shows a schematic block diagram illustrating an architecture of an apparatus according to an embodiment of the invention, which provides automatic sensor change in IMM-EKF.

FIG. 2 shows a schematic block diagram for illustrating an automatic sensor change principle according to an embodiment of the invention.

FIG. 3 shows diagrams illustrating variations from Normal distribution for two different driving cases.

FIG. 4 shows a diagram illustrating an estimated probability density function on all sensors according to an implementation example of the invention in which 14 sensors are used.

FIG. 5 shows a diagram illustrating a 20 seconds slice of pulsation estimation on a driving case, for all sensors, without selection of sensors.

FIG. 6 shows a schematic block diagram illustrating a model of a classifying q-Hurst parameters into a sensor number according to an implementation example of the present invention.

FIG. 7 shows a schematic block diagram illustrating sensor preprocessing steps according to an embodiment of the invention.

FIG. 8 shows a diagram illustrating magnitude and phase response of a passband filter used in the preprocessing steps.

FIG. 9 shows a diagram illustrating a nonlinearly transformed sensor signal according to the preprocessing steps.

FIG. 10 shows a schematic block diagram illustrating an initial frequency estimation principle according to an embodiment of the invention.

FIG. 11 shows a diagram illustrating the ESPRIT frequency estimation principle.

FIG. 12 shows a diagram illustrating a clustering principle for initial frequency decision according to an implementation example of the invention.

FIG. 13 shows a schematic block diagram illustrating nonlinear fitting using neural networks.

FIG. 14 shows a diagram illustrating IMM-EKF processing.

FIG. 15 shows a diagram illustrating a pulsation estimation result, using the automatic sensor change according to an embodiment of the present invention.

### DETAILED DESCRIPTION OF THE INVENTION

According to the present invention, biological parameters such as heartbeat and/or respiratory rhythm of a subject on a support are estimated. By way of non-limiting example, the support may comprise a seat, e.g. at home or of a car, or a bed. The support preferably includes at least two sensors which measure a variation of pressure, e.g. piezoelectric sensors.

The support may further include at least one accelerometer. In case the support is implemented as a seat in a vehicle, accelerometers positioned on the seat act particularly as reference sensors for surrounding noise such as vibration noise or the like from the vehicle, and detect noise in three orthogonal directions.

While driving, body movements are frequent and can have large amplitude. As body movement is considered here the movement of all body parts, e.g. legs, hands, corpus, head, individually or simultaneously. The body movements can be classified into two categories: movements related to driving, and movements related to physiological or psychological condition.

Here it is assumed that at least two sensors, e.g. two piezoelectric sensors, are embedded in a support such as a seat and are not noisy or already de-noised.

A. Architecture

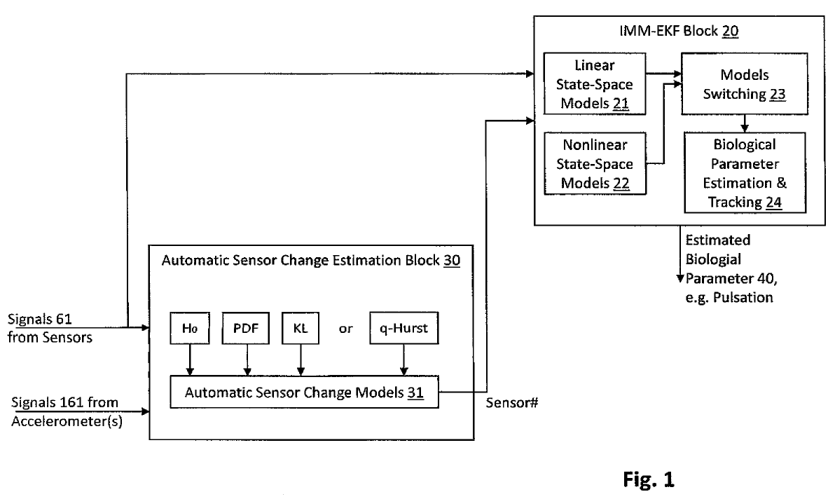

FIG. 1 shows a schematic block diagram illustrating an architecture of an apparatus according to an embodiment of the invention, that provides automatic sensor change in Interactive Multi-Model Extended Kalman Filter.

It is to be noted that signals and functions described in the embodiment are present in the digital domain.

The apparatus 1 shown in FIG. 1 comprises two blocks:
(1) an automatic sensor change estimation (ASCE) block 30 which predicts and selects the most probable best sensor to be used for pulsation estimation at a given time; and
(2) an interactive multi model extended Kalman filter (IMM-EKF) block 20 which switches linear/nonlinear state-space models 21, 22 when a new sensor is selected.

Signals 61 e.g. from sensors of a support (not shown) such as a seat in a vehicle (e.g. top seat piezo sensors and bottom seat piezo sensors) are processed by the apparatus 1. The signals 61 are digitized sensor (e.g. piezoelectric sensor) outputs that are noise reduced. The apparatus 1 may also process signals 161 e.g. from accelerometers comprised by the support, e.g. the seat in the vehicle. The signals 161 are digitized outputs from the accelerometers.

In this example embodiment, in the IMM-EKF block 20 only one sensor at every time frame is used. Therefore, assuming that there are N sensors, the ASCE block 30 selects one of the N sensors for a given time frame T. T may be in the order of 500 ms, one second or 2 s, etc.

The apparatus 1 represents an embodiment of the invention, for estimating a biological parameter 40 of a subject on a support, the support comprising N (N>=2) sensors each measuring a variation of pressure. M (M>=1) accelerometers are connected to the support. The apparatus 1 comprises linear/nonlinear state-space models (sensor-specific model) 21, 22 for each of the N sensors based on signals 61 from the N sensors, which will be described in more detail later on. The signals 61 corresponding to the variation of pressure measured by the N sensors, respectively. In a selection process, the ASCE block 30 selects, at every time frame T, one sensor out of the N sensors, to be used for estimating the biological parameter 40 of the subject in the IMM-EKF block 20, based on the signals 161 from the M accelerometers or the signals 161 and the signals 61, which will be described in more detail later on. In an estimation process, the IMM-EKE block 20 estimates the biological parameter 40 of the subject using in the biological parameter estimation & tracking block 24, at every time frame T, the sensor-specific model 21, 22 provided for the selected one sensor and switched by the models switching block 23, which will be described in more detail later on.

A block H₀ in the ASCE block 30 comprises a test if a noise distribution is a Normal distribution (Gaussian distribution). A block PDF in the ASCE block 30 comprises a probability density function which is a function that represents the relative likelihood for a random variable, here noise. A block KL in the ASCE block 30 comprises the Kullback-Leibler divergence, and a block q-Hurst in the ASCE block 30 comprises q^{th} order Hurst parameters according to the Random Multifractal theory.

1. The Automatic Sensor Change

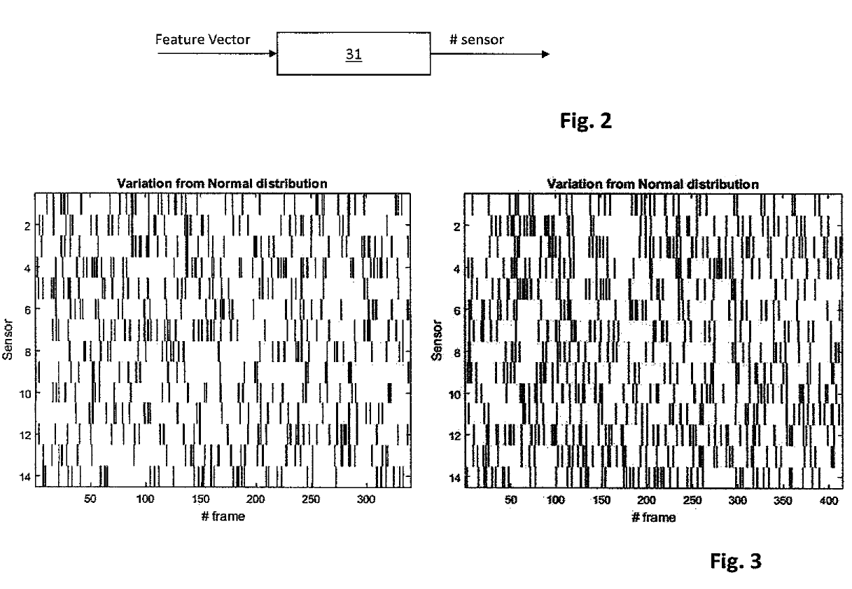

A principle of automatic sensor change according to an embodiment of the invention is shown in FIG. 2.

At every time frame T, feature parameters are computed for every accelerometer signal 161 or accelerometer signals 161 and sensor signals 61. The accelerometers measure vibration noise and are also influenced by some body movements.

The feature vector enters into a model of automatic sensor change models 31 of the ASCE block 30, and finally a decision is made about a sensor number (sensor#) to be used in the estimation process in the IMM-EKF block 20.

1.1 The Feature Vector

The feature vector is a set of parameters extracted from the accelerometers and sensors. The type of parameters is not limited. For example, the following parameters can be used:

a) q-Hurst Parameters

These parameters are based on the Random Multi-Fractal theory. The idea behind is to find a particular structure that is invariant of noise and sensor signal. The q-order Hurst exponents are used to parameterize the multifractal structure of the accelerometer signals 161 and sensor signal 61. The q-Hurst parameters are computed using multifractal de-trended fluctuation analysis.

A first operation is to integrate the signal. This is done by a cumulative sum

$${x}_{int} ( k ) = {\sum }_{l = 1}^{k} {x}_{k}$$

where xᵢₙₜ is the integrated accelerometer or sensor signal and x is the accelerometer or sensor signal.

The average amplitude variation, i.e. Root Mean Square (RMS), is computed using

$$F ( s , v ) = \sqrt{\frac{1}{s} {\sum }_{i = 1}^{s} {( {x}_{int} ( ( v - 1 ) s + i ) - {x}_{fit} ( i ) )}^{2}}$$

where F is the RMS value for scale s and segment index v. And is the quadratic de-trending of xᵢₙₜ given by

$${x}_{fit} ( i ) = {\sum }_{k = 0}^{2} {a}_{k} {i}^{2 - k}$$

where a comprises the fitting coefficients.

Generalizing, with q parameters, the multifractal amplitude variation is obtained as:

$${F}_{q} ( s ) = {\{ \frac{1}{2} {\sum }_{v = 1}^{2} {F ( s , v )}^{2 - q / 2} \}}^{1 / q}$$

Then the q-Hurst parameter is obtained by estimating the slope of log₂F_{q}(s). The “q” in Hurst parameter is an additional scale that when q is negative, segments with very small fluctuation are amplified, and when q is positive, segments with very large fluctuation are amplified.

Computation is therefore done for several scales s, for example s=4, 8, 16, 32, 64, 128, 256, 512. This is repeated for every desired q value, for example q=−5, −1, 5.

Since the q-Hurst parameter is the slope, and if there are 3 values for q, one possible feature vector will have a length of N*3, depending of the number of sensors. For example, if the sensors are subjected to the computation and there are 14 sensors in the seat, the feature vector input into model 31 is of length 42. By using the above feature vector it is possible to decide about the best sensor, i.e. the sensor to be used for the biological parameter estimation.

b) Statistical Parameters

One other possibility is to combine statistical descriptors to create the feature vector. Such computations are also done for each time frame T.

Null-Hypothesis (H₀) Test that the Distribution comes from a Normal Distribution

This computation is done for all sensors or accelerometers and comprises the Lilliefors test for Normality. If the data distribution is Normal then H₀=0 and H₀=1 otherwise.

A first step is the computation of mean and standard deviation values of the sensor signals 61 and/or the accelerator signals 161, which are given by:

$$\overline{x} = \frac{1}{L} {\sum }_{k = 1}^{L} {x}_{k} ; σ = \sqrt{\frac{1}{L - 1} {\sum }_{k = 1}^{L} {( {x}_{k} - \overline{x} )}^{2}}$$

where xₖ is the kth data sample of the current time frame. The time frame has L samples. and σ are x̅ spectively the sample mean value and the sample standard deviation value.

A second step computes the normalized value, for all samples of the frame:

$${Z}_{k} = \frac{{x}_{k} - \overline{x}}{σ}$$

Then, the Lilliefors test LF is computed by

LF=sup|F(x)−S(x)|

where LF is the supremum of the absolute value of the difference between the zero-mean Normal distribution with variance 1 (F(x)) and the empirical distribution of the Zₖ values. The test is rejected if LF is greater than the critical value for the test (the critical values are given by a table).

Hence, a measure of the variability of the noise probability distribution compared to the Normal probability distribution is derived. This comparison is made at every time frame.

FIG. 3 shows the variations when the noise probability density on the sensor has a Normal distribution or not. The x-axis is the time in frame number and the y-axis is the sensor number. The white color indicates when the distribution is Normal and the black color indicates when it is different.

The Probability Density Function (PDF)

The previous descriptor shows that the noise distribution not often is Gaussian, and therefore it is interesting to add a descriptor which details the shape of the distribution for all accelerometers or sensors.

The PDF can be computed by taking the histogram of the data of the accelerometer or sensor signals, or preferably using a kernel density estimation approach which converges to the true PDF, and is given by:

$$\hat{f} ( x , h ) = \frac{1}{Lh} {\sum }_{k = 1}^{L} K ( \frac{x - {x}_{k}}{h} )$$

where h is the bandwidth and K is the kernel. For example, the kernel can be Normal, Uniform, Epanechnikov, etc.

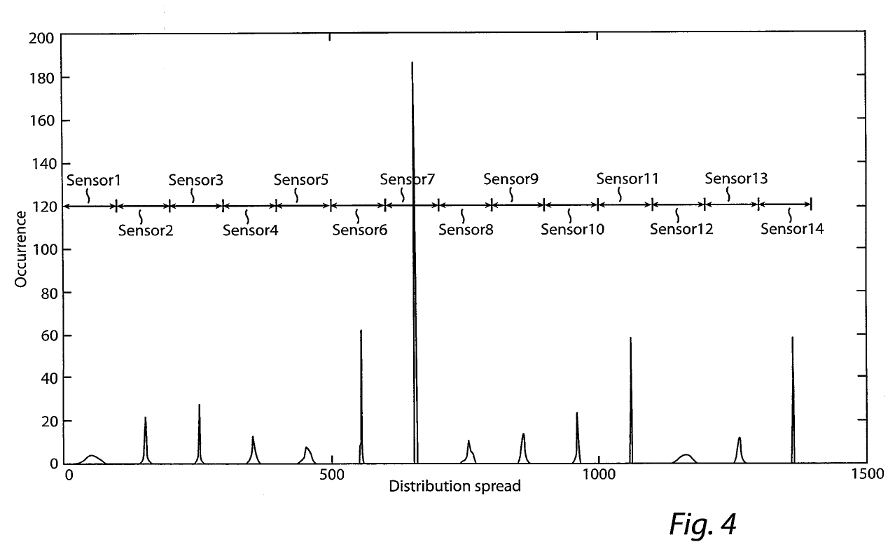

FIG. 4 shows for a given time instant, the estimates of the noise probability 5 density functions. All fourteen (14) sensors are placed on the horizontal axes, and every sensor limit is indicated by an arrow on the graph.

As can be seen from FIG. 4, the distributions are different at every sensor. Some are flat (e.g. sensor #12), some are heavy-tailed (e.g. sensor #7), some are dissymmetric (e.g. sensor #1, #4 and #8), etc. These distributions also change at every time step and therefore give an essential information on what is happening on sensors or accelerometers.

The Kullback-Leibler (KL) Divergence Cross PDFs

The PDF estimates at every time instant have been obtained. It may be also important to have a measure of the variation of the distribution on a given sensor compared to other sensors. Therefore, at a given time the KL divergence is computed for all sensors, cross all sensors at time

The KL divergence is given by:

$${KL}_{n} ( f , h ) = {\sum }_{i} {f}_{i} ln ( \frac{{f}_{i}}{{h}_{i}} )$$

where f and h are the 2 PDF to compute the divergence.

Therefore, if there are fourteen (14) sensors, there are 14*14=196 values computed at a given time.

Additional Statistical Parameters

In addition, the 3^{rd} and 4^{th} statistical moments can be computed, i.e. skewness and kurtosis, respectively, and added to the feature vector. They are given by

$$skewness = \frac{\frac{1}{L} {\sum }_{k = 1}^{L} {( {x}_{k} - \overline{x} )}^{3}}{{( \sqrt{\frac{1}{L} {\sum }_{k = 1}^{L} {( {x}_{k} - \overline{x} )}^{2}} )}^{3}}$$

$$kurtosis = \frac{\frac{1}{L} {\sum }_{k = 1}^{L} {( {x}_{k} - \overline{x} )}^{4}}{{( \sqrt{\frac{1}{L} {\sum }_{k = 1}^{L} {( {x}_{k} - \overline{x} )}^{2}} )}^{2}}$$

where all these parameters comprise the feature vector.

1.2 The Target

The target is the sensor number. Thus, the best probable sensor has to be decided for every time frame, e.g. by first checking the pulsation estimation on every sensor.

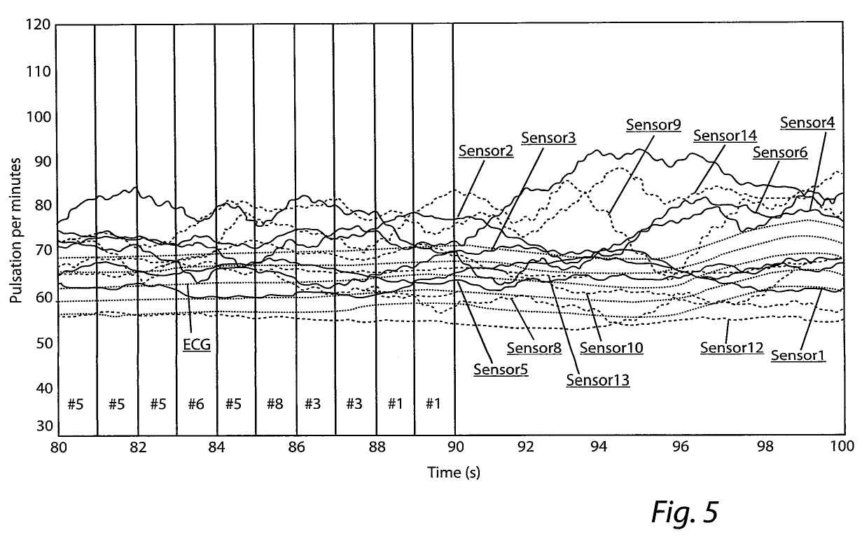

FIG. 5 shows one method to find the best sensor for a given subject and run. A 20 seconds zoom on the pulsation estimation for all sensors individually using an Extended Kalman Filter is shown. The time is split into segments of one second (here only the 10 first seconds are shown), and for each segment the best sensor is given (#5 means sensor number 5, etc.). FIG. 5 also shows an ECG 15 signal as a reference signal. Therefore the model 31 of FIG. 2 performs a nonlinear mapping between the feature vector at the input and the target at the output.

1.3 The Model

A nonlinear model is used based on Neural Networks (NN), because NNs can model any type of nonlinearity. The structure of the Neural Network that is used to perform the classification (i.e. decision about the most probable best sensor) is shown in FIG. 6 which illustrates an example of the structure of the classifier (i.e. decision) model 31, when the input feature vector is based on three q-Hurst parameters per sensor. It is to be noted that the input feature vector is not limited to only three q-Hurst parameters, and more than three parameters can be used.

As also depicted in FIG. 6, forty-two (42) parameters are present at the input which correspond to three q-Hurst parameters per sensor, and 14 parameters are present at the output. Each element in the output corresponds to a sensor number: element 1 corresponds to sensor #1, element 2 corresponds to sensor #2, etc.

The sensor number decision is the element of the output which has the maximum value, i.e. highest probability. The structure is ready for other type of decision, since there is a probability given, at every second, for every sensor number. Most preferably, the structure is adapted to the type of input feature vector.

The structure shows a hidden layer and an output layer. The number of neurons in the hidden layer depends on the structure and the input parameters (feature vector), for example 250 neurons, but can be whatever value that achieves a reliable sensor number prediction. The hidden layer contains a weight (w), a bias (b) and a nonlinear function, a sigmoid in this case (for one neuron). The output layer contains a weight (w), a bias (b) and a nonlinear function, here a softmax function.

The sigmoid and softmax equations used are given below:

$${y}_{sigmoid} = \frac{2}{1 + {e}^{- 2 x}} - 1 ; {y}_{softmax} = \frac{{e}^{x}}{Σ {e}^{x}}$$

where the softmax function provides a measure of certainty (i.e. posterior probability).

At first, data has to be prepared for the computation of the parameters. This is described below.

$$\begin{matrix}\underline{Input} & \underline{Target} \\ 42 \underbrace{\{ [ \begin{matrix}{H}_{s 1 , 1 , t = 0} & … & {H}_{s 1 , 1 , t = N} \\ {H}_{s 1 , 2 , t = 0} & … & {H}_{s 1 , 2 , t = N} \\ {H}_{s 1 , 3 , t = 0} & … & {H}_{s 1 , 3 , t = N} \\ ⋮ & … & ⋮ \\ {H}_{s 14 , 1 , t = 0} & … & {H}_{s 14 , 1 , t = N} \\ {H}_{s 14 , 2 , t = 0} & … & {H}_{s 14 , 2 , t = N} \\ {H}_{s 14 , 3 , t = 0} & … & {H}_{s 14 , 3 , t = N}\end{matrix} ]}_{C} & 14 \underbrace{\{ [ \begin{matrix}0 & … & 0 \\ 0 & … & 1 \\ 0 & … & 0 \\ 1 & … & 0 \\ 0 & … & 0 \\ 0 & … & 0 \\ 0 & … & 0 \\ 0 & … & 0 \\ 0 & … & 0 \\ 0 & … & 0 \\ 0 & … & 0 \\ 0 & … & 0 \\ 0 & … & 0 \\ 0 & … & 0\end{matrix} ]}_{C}\end{matrix}$$

The input matrix should have forty-two (42) lines corresponding to the number of q-Hurst parameters of the for all 14 sensors, and C columns which correspond to the number of seconds of data given per parameter. Each new column is a different time instant, as explained previously. The target matrix has the same number of columns as the input matrix, however there are only 14 lines, each one corresponding to a sensor. Therefore, only the corresponding selected best sensor has to be set to one and other values must be zero.

The parameters of the model 31 can then be computed. This process is also called training, because it is an iterative computation and the same target value can have different input values. This computation is done using the scaled conjugate gradient backpropagation approach. This same computation method can hold for a different feature vector, like the statistical descriptors feature vector.

Once the parameters of the nonlinear model 31 are computed, it can be used to decide the sensor to be used for pulsation estimation.

Then, at every time frame, the q-Hurst parameters are computed, or the statistical descriptors are computed, for the sensors or accelerometers, and the feature vector x_{feature} of parameters is created. This vector is used in the following computations. First the input should be mapped by using

yₘₐₚ(k)=(x_{feature}(k)−x_{offset}(k))*G(k)−1

where x_{offset} is the offset to be removed from the feature vector and G is the gain. These offsets and gains were computed in the previous offline procedure.

Then, the output of the hidden layer is computed, which is

y_{hidden}=y_{sigmoid}(B_{hidden}+W_{hidden}yₘₐₚ)

where B_{hidden} is the bias vector of the hidden layer which has a length equal to the number of neurons in the hidden layer, Q, W_{hidden} is a matrix of coefficients of the hidden layer, that is of size [Q×P], P is the length of the feature vector, and y_{sigmoid} is the nonlinear sigmoid function where the equation was given earlier. The output of the hidden layer is a vector of length Q.

The last step is the computation of the output of the output layer, which is given by the following equation:

yₒᵤₜ=y_{softmax}(Bₒᵤₜ+Wₒᵤₜy_{hidden})

where Bₒᵤₜ is the bias vector of the output layer which has a length equal to the number of sensors N, Wₒᵤₜ is a matrix of coefficients of the output layer, that is of size [N×Q], and y_{softmax} is a the nonlinear softmax function for which the equation was given earlier. The output yₒᵤₜ is a vector of length equal to the number of sensors, i.e. N. The best probable sensor is then the number of the element corresponding to the largest value in yₒᵤₜ.

It should be noted that neural networks are not the only possibility, and can be replaced by Hidden Markov Models. The sensor number is communicated to the IMM-EKF block 20.

2. The Robust Pulsation Estimation with IMM-EKF

An Extended Kalman Filter (EKF) is known to those of skill in the art. For example, an EKF is described in French Patent Application Publications FR 2,943,233 A1, FR 2,943,234 A1 and FR 2,943,236 A1, incorporated herein by reference.

In this example embodiment, since only one sensor is kept at a time for pulsation estimation, and it is changed in time, when it is necessary using the previously described approach for Automatic Sensor Change, the Kalman Filter needs to be adapted to switch observation models and state-space models, when the condition requires it.

The role of the IMM-EKF block 20 is to estimate the pulsation in a robust way, regardless of vibration noise and body movements, e.g. if contact with sensor remains. The IMM-EKK block 20 is an extended version of the EKF that can switch between models. Basically, it consist of three main steps: mixing, filtering and combination.

The decision of changing the model is based mainly on mixing probabilities. An EKF estimation is executed on every model in this example embodiment. Since, in this example, information on a sensor number to be used for estimation is provided, the original IMM-EKF approach is modified to use such information. Since there is the possibility to switch between N sensors, there are provided N state-space models and N observation models (linear state-space models 21 and nonlinear observation models 22).

2.1 Preprocessing of Piezo Sensors

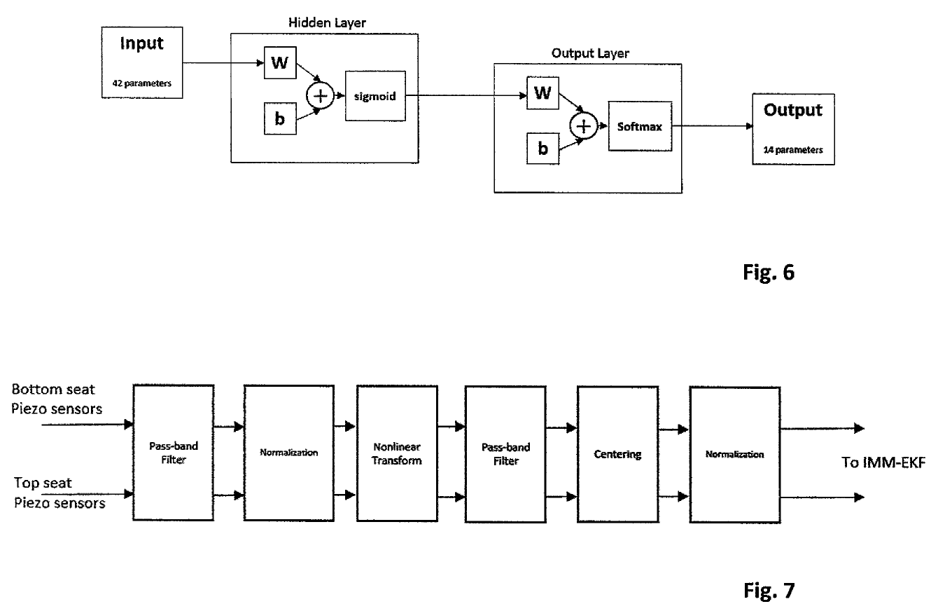

Prior to using the sensor signals 61 in the IMM-EKF block 20, which are noise reduced but still contain some noise, a preprocessing is carried out in order to maximize the estimation performance, FIG. 7 illustrates preprocessing steps for the sensor signals 61 (e.g. piezo signals comprising bottom seat piezo sensors and top seat piezo sensors).

Pass-Band Filter

First, the sensor signals 61 are subjected to pass-band filtering. The pass-band filter used for this purpose is centered on frequencies of interest for the pulsation estimation. An Infinite Impulse Response (IIR) filter has been designed having the following characteristics:

Center frequency: f₀=1.3 Hz

Pass-band: b=2.5 Hz

Order: 3

To design such a filter, the following computations are done:

w₀=πb; w₁=2πf₀

and the following vectors are defined:

$${B}_{0} = [ \begin{matrix}2 \frac{{w}_{0}}{{w}_{1}} & 0\end{matrix} ] ; {A}_{0} = [ \begin{matrix}1 & 2 \frac{{w}_{0}}{{w}_{1}} & 1\end{matrix} ]$$

Then, since the filter order is 3, these values are convolved twice in the following way:

B=B₀; A=A₀

B=B*B₀ (to be done twice)

A=A*A₀ (to be done twice)

where “*” is the symbol for convolution (and not multiplication).

Then, since an IIR filter has been designed, the bilinear transform is used to move the design to the Z-domain. For the bilinear transform we use the warp frequency as

$$w = \frac{1}{2} {tan}^{- 1} ( \frac{{w}_{1}}{2 {f}_{s}} )$$

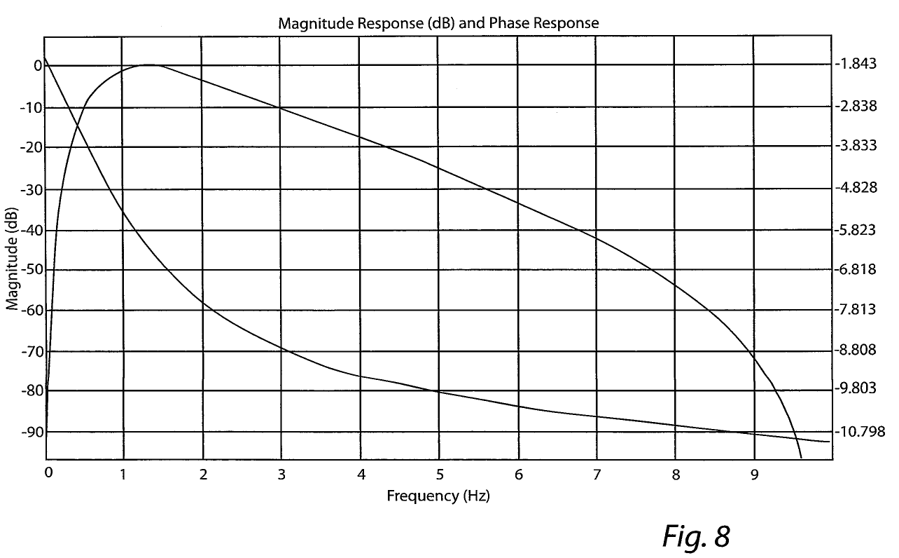

The characteristics of the filter are shown in FIG. 8, where the magnitude response is shown in solid lines and the phase response of the pass-band filter is shown in broken lines.

b) Normalization

The pass-band filtered signal then is subjected to normalization. This first normalization divides the sensor signals by the standard deviation of the first frame (i.e. five seconds).

$${σ}_{i} = std ( {S}_{i \vert 0 < t < {T}_{frame}} ) ; {S}_{n , i} = \frac{{S}_{i}}{{σ}_{i}}$$

where S_{n,i} is the normalized signal of sensor i, Sᵢ is the sensor signal S_{t|0<t}_{_{tframe}} is the frame sample of the sensor i, and std stands for standard deviation which is σ.

c) Nonlinear Transform

The vibrations and body movements cause large variations of amplitude in the sensor signal depending the situation. These large variations of amplitude may have an impact on the pulsation estimation. Therefore, a nonlinear transformation step aims to provide a signal that has constant amplitude but where oscillations are kept.

The hyperbolic tangent is used as the nonlinear transform function, and the signal after nonlinear transformation is computed to:

$${Y}_{NL , i} = \frac{tanh \frac{{S}_{n , i}}{1.4 σ}}{1.4 σ}$$

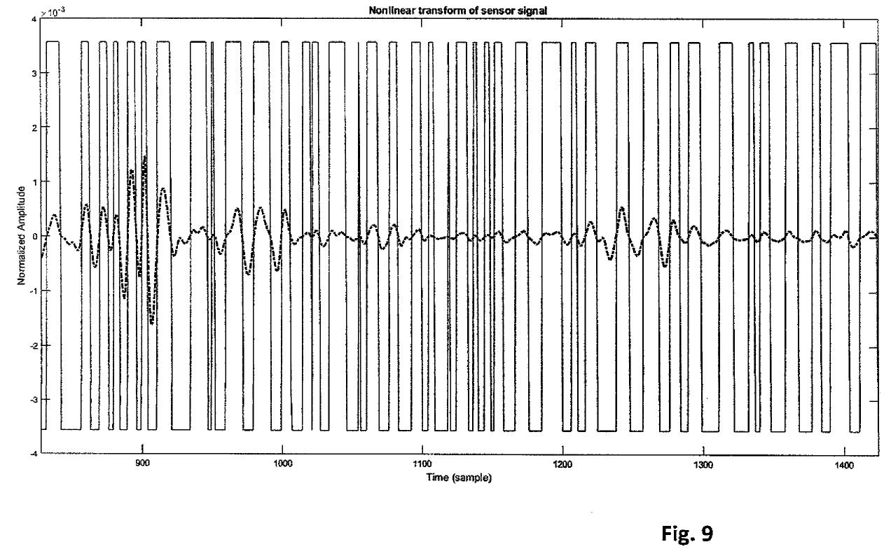

FIG. 9 shows the result on the nonlinear transform in solid line compared to the sensor signal before transformation shown in a broken line curve that is normalized in amplitude so that it can be compared with the transformed signal on the same figure. The real values of the sensor signal are approximately 10,000 times larger. As can be seen from FIG. 9, the amplitude fluctuations are substantially constant whatever variations are present at the input, and the oscillation periods are kept stable.

d) Pass-Band Filter

The same pass-band filter as previously explained in section a) is applied after the nonlinear transformation. This results into a more sinusoidal shape of the 5 signal.

e) Centering

Then, after applying the pass-band filtering again, the following operation of centering represents the removal of the sample mean value from the signal:

$${Y}_{c , i} = {Y}_{bp , i} - \overline{{Y}_{bp , l}} ; \overline{{Y}_{bp , l}} = \frac{1}{N} {\sum }_{k = 1}^{N} {Y}_{bp , i , k}$$

where Y_{c,i} is the centered signal, Y_{bp,i} is the band-passed signal as processed in d), and Y̅_{bp,i} is the sample mean value.

1) Normalization

This last step is the final normalization of the preprocessed sensor signal. It is a normalization by the standard deviation value over the signal duration, or the current frame T:

$${σ}_{2 , i} = std ( {Y}_{c , i} ) ; {Y}_{n , i} = \frac{{Y}_{c , i}}{{σ}_{2 , i}}$$

2.2 Initialize State Parameters

State parameters of the IMM-EKF block 20 are set into a vector form for the ease of the estimation procedure. Since there are N sensors, there are N models 21, 22, and there are also N state vectors that will be used for estimation. As mentioned before, N may be equal to 14 for example, but is not limited thereto.

The parameters to be estimated are the frequency f, amplitude A and phase φ. The state vectors are then:

$${\hat{x}}_{i} = [ \begin{matrix}{f}_{i} \\ {A}_{i} \\ {ϕ}_{i}\end{matrix} ] ; i = 1 … N$$

The N state vectors \hat{x}ᵢ have 3 parameters to be initialized in the IMM-EKF block 20. For example, these 3 parameters are estimated using the first 1.5 seconds of the sensor signals. Although these parameters can be filled randomly, the convergence time may be longer in case of no good choice.

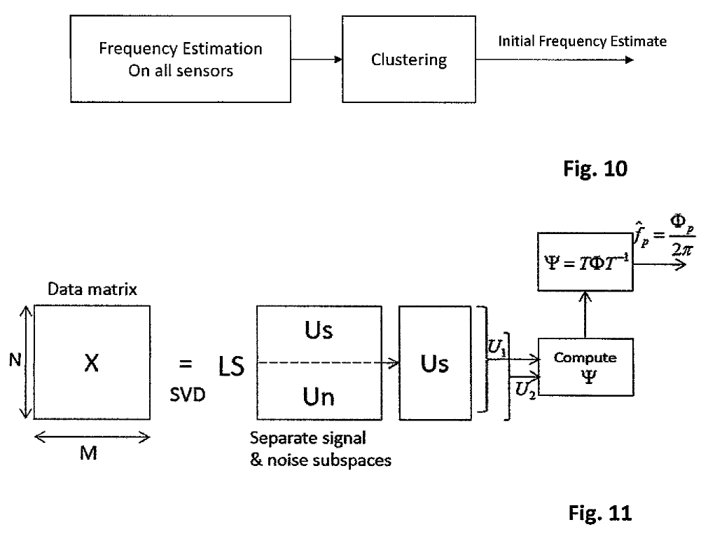

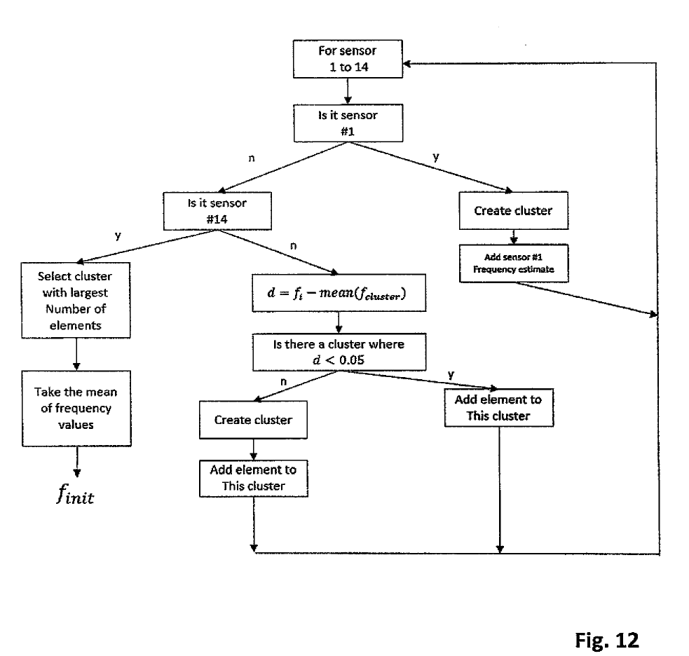

FIG. 10 shows that the estimation of the frequency has two steps: the frequency estimation itself on all sensors and the decision about the value to be used. The frequency estimation uses a subspace approach, meaning decomposition of the signals in eigenvalues and eigenvectors. The ESPRIT (Estimation of Signal Parameters via Rotational Invariance Techniques) estimator is used. The principle is described in FIG. 11.

The ESPRIT frequency estimator uses a deterministic relationship between subspaces. The first operation to be done is to build a data matrix X. This is done in the following way:

$$X = [ \begin{matrix}x ( 1 ) \\ x ( 2 ) \\ ⋮ \\ x ( 0 )\end{matrix} ] = [ \begin{matrix}x ( 1 ) & x ( 2 ) & … & x ( D ) \\ x ( 2 ) & x ( 3 ) & … & x ( D + 1 ) \\ ⋮ & ⋮ & ⋮ & ⋮ \\ x ( 0 ) & x ( 0 + 1 ) & … & x ( 0 + D )\end{matrix} ]$$

where x is data of the sensor signal, and D is a window size. For example, D=8 . “O” is the number of samples used.

Then, on the X matrix the Singular Value Decomposition is applied (SVD), and X can be rewritten as X=LSU, where L is a [O×O] matrix of left singular vectors and U is a [D×D] matrix of right singular vectors. S is a [O×D] matrix of singular values on the main diagonal ordered in descending magnitude.

U forms an orthonormal basis for the Multi-dimensional vector space. This subspace can be partitioned into signal (Uₛ) and noise (Uₙ subspaces. The threshold between subspaces is set to P=5. This means that Uₛ is the matrix of right-hand singular values with the P largest magnitudes.

The next step is to stagger subspaces by separating them into U₁ and U₂. U₁ contains the element from 1 to D-1, and U₂ contains the elements from 2 to D. The rotational property is between staggered subspaces and this produces the frequency estimates.

Then, ψ is computed as:

Ψ=(U₁^{T}U₁)⁻¹U₁^{T}U₂

The frequency estimates are then given in FIG. 11, where contains the eigenvalues of ψ. Once this is done for all sensor signals, then the decision is made after clustering the frequency estimation values obtained on each sensor.

As shown in FIG. 12, the clustering is iterated for all sensor frequency estimates (14 sensors in this case) that was obtained by the previous frequency estimation procedure. Basically, if the distance d is closer to the gravity center of the cluster (the mean value of all frequency estimates in the cluster), then the new frequency estimate is added to this cluster. Otherwise, a new cluster is created. Finally, the cluster which has the largest number of elements is selected and the initial frequency is the mean value of the frequency estimates in this cluster.

The amplitude and phase can be set to zero, without influencing the pulsation estimates. If a precise estimate is desired, this can be achieved by using the Least-Square parameter estimation. These values are set for all N=14 state vectors, in this example.

2.3 Initialize Process and Noise Covariance

The process noise is the noise related to the state parameters. There are two parameters where the process noise is needed to be defined. The frequency estimate noise d_{f} and the amplitude estimate noise d_{A}.

Amplitude estimate noise d_{A} is not of very high influence for the IMM-EKF pulsation estimation. It is simply set (e.g. found empirically) to d_{A}=5.10⁻¹¹. Frequency estimate noise d_{f} is of high influence in the IMM-EKF and will enable good tracking of the pulsation or very bad tracking if mistaken. The value of d_{f} is computed online based e.g. on the first 1.5 second of the signal from each sensor. This value might be different on each sensor.

A nonlinear model was found between sensor covariance values and optimum values for d_{f} based on the computation of the Cramer-Rao Lower Bound. These optimum values can be easily found empirically, by setting different values for d_{f} and checking the results achieved by the EKF when estimating the pulsation. The best pulsation estimation leads to the best d_{f} values. In other words, in this example a model is sought for fitting the input (observation covariance) and the known optimum d_{f} values.

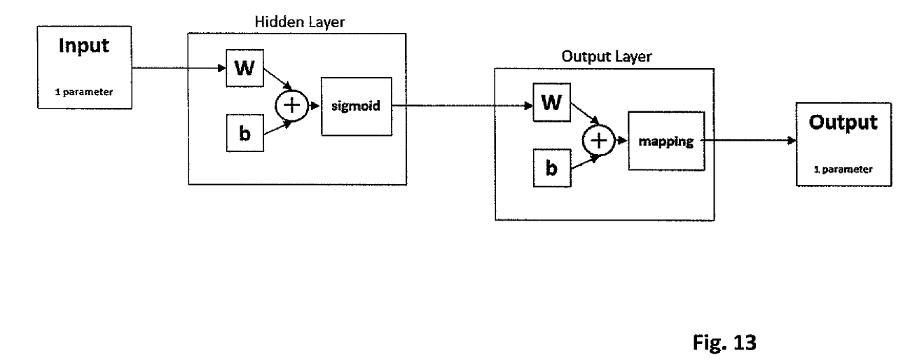

If a polynomial fitting is used, the error will be relatively high. With neural networks complex nonlinearities can be modeled. FIG. 13 illustrates nonlinear fitting using neural networks. This neural network structure is slightly different from that shown in FIG. 6 used for automatic sensor change. Here, the output layer is just a mapping. The output layer has one neuron and the hidden layer has 15 neurons. The input and output has only one parameter. The computation of model parameters is done using the Levenberg-Marquardt approach.

Once the model is computed then it can be used online e.g. during the first 1.5 seconds of the sensor signals. The computation is then very similar to the automatic sensor change, and is given below:

yₘₐₚ=(R−x_{offset})*G−1

where x_{offset} is the offset to be removed from the observation covariance R and G is the gain. These offsets and gains were computed in the previous offline procedure.

Then, the output of the hidden layer is computed, which is

y_{hidden}=y_{sigmoid}(B_{hidden}+W_{hidden}yₘₐₚ)

where B_{hidden} is the bias vector of the hidden layer which has a length equal to the number of neurons in the hidden layer, Q.

W_{hidden} is a vector of coefficients of the hidden layer, that is of size [Q×1]. y_{sigmoid} is the nonlinear sigmoid function where the equation was given earlier. The output of the hidden layer y_{hidden} is a vector of length M.

The last step is the computation of the output of the output layer, which is given by the following equation:

$${d}_{f} = \frac{( {B}_{out} + {W}_{out} {y}_{hidden} ) + 1}{G} + 10$$

where Bₒᵤₜ is the bias vector of the output layer which has a length equal to 1. Wₒᵤₜ is a vector of coefficients of the output layer, that is of size [1×Q]. The output d_{f} is to be directly used in the IMM-EKF block 20. The remaining noise covariance estimate is the variance of the sensor signal in the current time period.

2.4 IMM-EKF Estimation

There are N state vectors named \hat{x}₁ to \hat{x}_{N} and the 3 parameters, frequency, amplitude and phase, that are different for all models 21, 22. The IMM-EKF general equations for all sensors are given by:

xₖⁱ=Fₖ₋₁xₖ₋₁¹+vₖⁱ

yₖⁱ=hₖ₋₁(xₖⁱ)+wₖⁱ

where is the linear state space equation at the sample k, and yₖ is the nonlinear observation equation. vₖ and wₖ are respectively the process and observation noises. The index i is the sensor number. Fₖ₋₁ is the linear state-space model at sample time k-1, and hₖ₋₁ i is the nonlinear observation model at sample time k-1.

F is the linear state-space transition matrix and in our case is equal to:

$$F = [ \begin{matrix}1 & 0 & 0 \\ 0 & 1 & 0 \\ \frac{2 π}{fs} & 0 & 1\end{matrix} ]$$

The state-space matrix may be changed during the model switching by the models switching block 23 shown in FIG. 1, but in the described case with the Hurst automatic sensor change, this is not necessary.

For example, the observation equation can be rewritten as:

yₖⁱ=Aₖⁱ sin φₖⁱ+wₖⁱ

The observation equation may be a sum of sinewaves as described in French Patent Application Publications FR 2,943,233 A1, FR 2,943,234 A1 and FR 2,943,236 A1, incorporated herein by reference. The IMM-EKF has three steps, and their equations are given below:

Prediction:

\hat{x}_{k|k-1}ⁱ=F\hat{x}_{k-1|k-1}ⁱ

P_{k|k-1}ⁱ=Qⁱ+FP_{k-1|k-1}ⁱF^{T}

where \hat{x}_{k|k-1} is a prediction of a current state parameters knowing the previous parameters, \hat{x}_{k-1|k-1} are the previous state parameters, P_{k|k-1} is the prediction of the covariance knowing the previous covariance, and Q is the process noise covariance.

Update:

\hat{x}_{k|k}ⁱ=\hat{x}_{k|k-1}ⁱ+Kₖⁱ(yₖⁱ−hₖ(x_{k|k-1}ⁱ))

P_{k|k}ⁱ=P_{k|k-1}ⁱ−KₖⁱSₖⁱKₖ^{T}^{ⁱ}

where

Sₖⁱ=\tilde{H}ₖⁱP_{k|k-1}ⁱ\tilde{H}ₖ^{T}^{ⁱ}+Rⁱ

Kₖⁱ=P_{k|k-1}ⁱ\tilde{H}ₖ^{T}^{ⁱ}Sₖ⁻¹^{ⁱ}

Since h is a nonlinear function, it needs to be linearized. Therefore, \tilde{H}ₖⁱ is the local linearization of the nonlinear function h for sensor i. It is defined as the Jacobian evaluated at \hat{x}_{k, k-1}ⁱ and in the case mentioned above, it results to:

$${\tilde{H}}_{k}^{i} = {[ {∇}_{{x}_{k}} {h}_{k}^{T} ( {x}_{k} ) ]}_{{x}_{k} = {\hat{x}}_{k} \vert k = 1}^{T} = \{ \begin{matrix}0 \\ sin {ϕ}_{k}^{i} \\ {Λ}_{k}^{i} cos {ϕ}_{k}^{i}\end{matrix}$$

Model Switching:

The state vector estimates for all sensors have been derived, and therefore depending on the decision of the automatic sensor change block 30, the final state parameters estimates are given as:

\hat{x}k|k=\hat{x}_{k|k}^{p}

P_{k|k}=P_{k|k}^{p}

where p is the sensor number predicted by the automatic sensor change block 30.

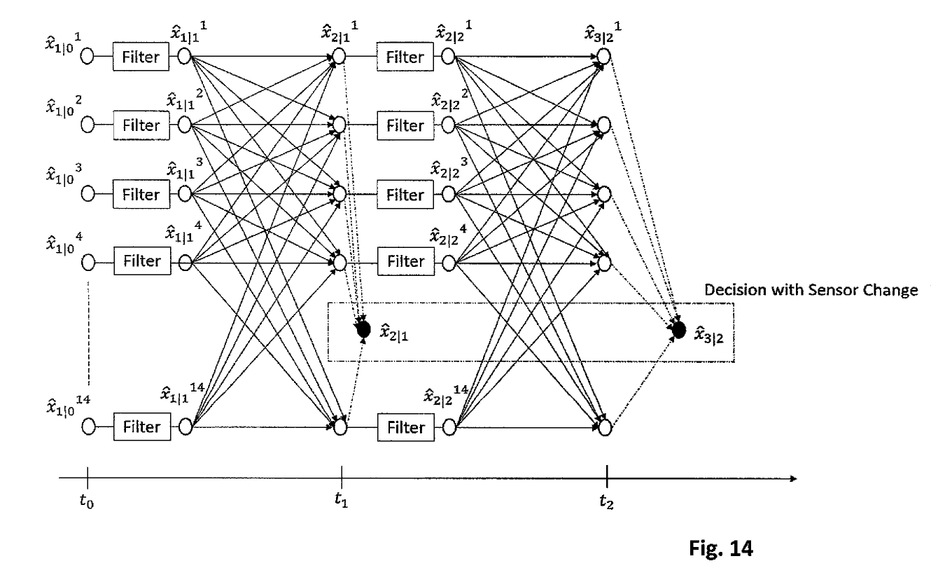

FIG. 14 shows the IMM-EKF processing of the IMM-EKF block 20 with the 14 models 21, 22 (when N=14) and where models can switch. According to the embodiment of the invention illustrated in FIG. 1, the models switching block 23 performs the switching of the models 21, 22. The black points are the final state estimates, which are used to provide the frequency estimate of the pulsation for the given time frame T by the biological parameter estimation & tracking block 24 shown in FIG. 1. The “Filter” block depicted in FIG. 14 corresponds to the update processing in the IMM-EKF block 20, which is performed by the biological parameter estimation & tracking block 24.

When the Automatic Sensor Change and the IMM-EKF are combined a robust pulsation estimation can be achieved even under strong body movement and in general for all body movements.

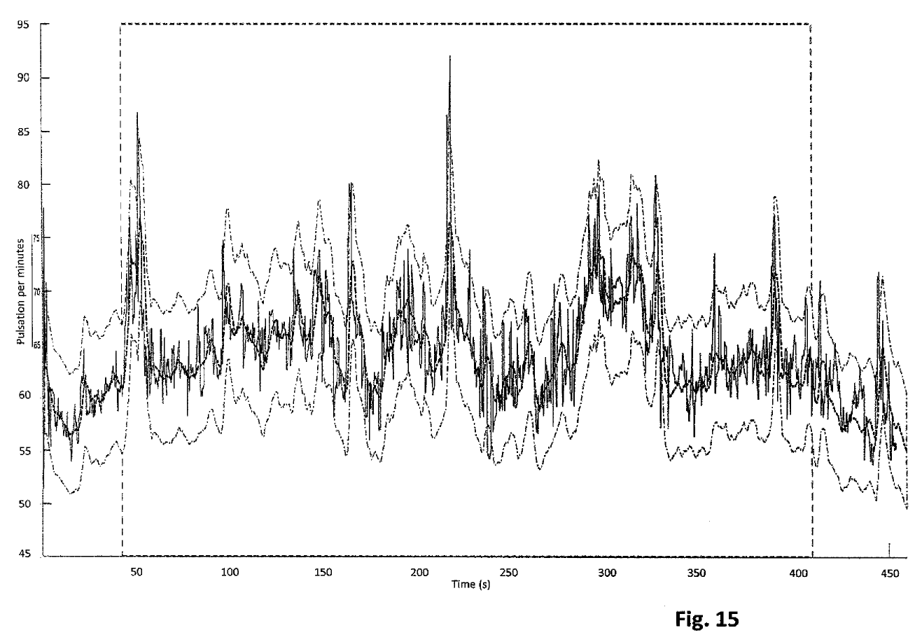

FIG. 15 shows the pulsation estimation result. The x-axis is the time and the y-axis is the number of pulsation per minute of the heart. The dashed box corresponds to the time where the car is moving (driving situation with high speed turns). The other area is where the car is static. 10⁰/0 tolerances are shown (external dash-dot curves). The dashed curve in the middle is the real pulsation value and the solid line is the estimation result. As can be seen from FIG. 15, the pulsation estimation can be very precise, and the oscillations that can be seen in the pulsation estimate result represent the breathing modulation of the pulsation.

In the present example, an automatic sensor change decision is provided that predicts, at every time frame T, one sensor (preferably the best sensor) to be used for the pulsation estimation depending on the noise and body movements. In addition, pulsation estimation using sensor change information is provided within an IMM-EKF that switches models, when it is judged necessary.

The functions of the apparatus 1 shown in FIG. 1 may be embodied in software, firmware and/or hardware, as is appropriate. In general, the embodiments of this 5 invention may be implemented by computer software stored in a memory and executable by a processor, or by hardware, or by a combination of software and/or firmware and hardware.

The memory may be of any type suitable to the local technical environment and may be implemented using any suitable data storage technology, such as semiconductor-based memory devices, magnetic memory devices and systems, optical memory devices and systems, fixed memory and removable memory. The processor may be of any type suitable to the local technical environment, and may include one or more of general purpose computers, special purpose computers, microprocessors, digital signal processors (DSPs) and processors based on a multi-core processor architecture, as non-limiting examples.

Further in this regard it should be noted that the various logical step descriptions above may represent program steps, or interconnected logic circuits, blocks and functions, or a combination of program steps and logic circuits, blocks and functions.

It is to be understood that the above description is illustrative of the invention and is not to be construed as limiting the invention. Various modifications and applications may occur to those skilled in the art without departing from the scope of the invention as defined by the appended claims.

What is claimed is:

1. A method for estimating a biological parameter of a subject on a support, the support comprising at least two sensors each measuring a variation of pressure, wherein at least one accelerometer is connected to the support, the method comprising:
providing a sensor-specific model for each of the at least two sensors based on signals from the at least two sensors, the signals corresponding to the variation of pressure measured by the at least two sensors, respectively; and
selecting, in a selection process, at every time frame T, one sensor out of the at least two sensors, to be used for estimating the biological parameter of the subject, based on signals from the at least one accelerometer; and estimating, in an estimation process, the biological parameter of the subject using, at every time frame T, the sensor-specific model provided for the selected one sensor.

2. The method of claim 1 wherein in the selection process the one sensor is selected based on the signals from the at least one accelerometer and the signals from the at least two sensors.

3. The method of claim 1 further comprising:
in the selection process, calculating a feature vector comprising parameters, for every time frame T, extracted from the signals from the at least two sensors and/or the signals from the at least one accelerometer, the parameters comprising q-Hurst parameters and/or statistical parameters, the statistical parameters comprising at least one of Null-Hypothesis parameters, Probability Density Function parameters, Kullback-Leibler divergence parameters, and skewness and kurtosis parameters;
wherein the feature vector is input into a non-linear model that maps the parameters of the feature vector, for every time frame T, into a number of the selected one sensor.

4. The method of claim 3 wherein the nonlinear model maps the parameters of the feature vector, for every time frame T, into a number of the selected one sensor by combining a nonlinear function and a decision based on probabilities.

5. The method of claim 1 further comprising:
in an initialization process for the estimation process, estimating, for each of the at least two sensors, state parameters of a state vector, the state parameters comprising an initial frequency, based on each of the signals from the at least two sensors.

6. The method of claim 5 further comprising:
in the initialization process, obtaining, for each of the at least two sensors, for at least one of the state parameters, a process noise value by using a nonlinear mapping model mapping covariance values to process noise values, wherein the process noise value is used in the estimation process.

7. The method of claim 6 wherein the mapping model maps a covariance value to a process noise value by a first calculation of a sigmoid function of a bias vector of a hidden layer of the nonlinear mapping model and a vector of coefficients of the hidden layer multiplied by a mapping value calculated from the covariance value, and a second calculation of a mapping function of a bias vector of an output layer of the nonlinear mapping model and a vector of coefficients of the output layer multiplied by the result of the first calculation.

8. The method of claim 5 further comprising:
in the estimation process, for each of the at least two sensors, calculating the state vector of a current sample (k) of the signal from the respective sensor based on the state vector of a previous sample (1<−1) of the signal, using the sensor-specific model for the respective sensor computed in the selection process.

9. The method of claim 8 wherein the process noise values which are obtained for the at least one state parameter of the at least two sensors are used for calculating the state vector.

10. The method of claim 8 wherein the calculating the state vector comprises:
predicting a current state vector of the current sample based on a previous state vector of the previous sample and a linear model of the sensor-specific model for the respective sensor; and
updating the predicted current state vector by using a non-linear model of the sensor-specific model of the respective sensor; and
based on the one sensor computed in the selection process, switching the sensor-specific model to be used for estimating the biological parameter to the sensor-specific model provided for the selected one sensor.

11. The method of claim 5 further comprising:
pre-processing the signals from each of the at least two sensors, wherein the pre-processed signals are output as the signals from the at least two sensors to the initialization process and the estimation process.

12. The method of claim 11 wherein the pre-processing comprises:
pass-band filtering the signals into first pass-band filtered signals;
normalizing the first pass-band filtered signals into first normalized signals;
non-linearly transforming the first normalized signals into transformed signals;
pass-band filtering the transformed signals into second pass-band filtered signals;
centering the second pass-band filtered signals into centered signals; and
normalizing the centered signals into the pre-processed signals.

13. A computer program product including a program for a processing device, comprising software code portions for implementing a process when the program is run on the processing device, the process comprising:
providing a sensor-specific model for each of the at least two sensors based on signals from the at least two sensors, the signals corresponding to the variation of pressure measured by the at least two sensors, respectively; and
selecting, in a selection process, at every time frame T, one sensor out of the at least two sensors, to be used for estimating the biological parameter of the subject, based on signals from the at least one accelerometer; and estimating, in an estimation process, the biological parameter of the subject using, at every time frame T, the sensor-specific model provided for the selected one sensor.

14. The computer program product according to claim 13 wherein the computer program product comprises a non-transitory computer-readable medium on which the software code portions are stored.

15. The computer program product according to claim 13 wherein the program is directly loadable into an internal memory of the processing device.

16. An apparatus for estimating a biological parameter of a subject on a support, the support comprising at least two sensors each measuring a variation of pressure, wherein at least one accelerometer is connected to the support, wherein a sensor-specific model is provided for each of the at least two sensors based on signals from the at least two sensors, the signals corresponding to the variation of pressure measured by the at least two sensors, respectively, the apparatus comprising:
a selecting unit configured to select, at every time frame T, one sensor out of the at least two sensors, to be used for estimating the biological parameter of the subject, based on signals from the at least one accelerometer; and
an estimating unit configured to estimate the biological parameter of the subject using, at every time frame T, the sensor-specific model provided for the selected one sensor.

17. The apparatus of claim 16, wherein the selecting unit is configured to select the one sensor based on the signals from the at least one accelerometer and the signals from the at least two sensors.

18. The apparatus of claim 16 wherein the selecting unit is configured to:
calculate a feature vector comprising parameters, for every time frame T, extracted from the signals from the at least two sensors and/or the signals from the at least one accelerometer, the parameters comprising q-Hurst parameters and/or statistical parameters, the statistical parameters comprising at least one of Null-Hypothesis parameters, Probability Density Function parameters, Kullback-Leibler divergence parameters, and skewness and kurtosis parameters, and
input the feature vector into a non-linear model for mapping the parameters of the feature vector, for every time frame T, into a number of the selected one sensor.

19. The apparatus of claim 18 wherein the selecting unit comprises the non linear model which is configured to map the parameters of the feature vector, for every time frame T, into a number of the selected one sensor by combining a nonlinear function and a decision based on probabilities.

20. The apparatus of claim 16 wherein the estimating unit is configured to, in an initialization process, estimate, for each of the at least two sensors, state parameters of a state vector, the state parameters comprising an initial frequency, based on each of the signals from the at least two sensors.

21. The apparatus of claim 20 wherein the estimating unit is configured to, in the initialization process, obtain, for each of the at least two sensors, for at least one of the state parameters, a process noise value by using a nonlinear mapping model for mapping covariance values to process noise values, wherein the process noise value is used in the estimation process.

22. The apparatus of claim 21 wherein the estimating unit comprises the nonlinear mapping model which is configured to map a covariance value to a process noise value by a first calculation of a sigmoid function of a bias vector of a hidden layer of the nonlinear mapping model and a vector of coefficients of the hidden layer multiplied by a mapping value calculated from the covariance value, and a second calculation of a mapping function of a bias vector of an output layer of the nonlinear mapping model and a vector of coefficients of the output layer multiplied by the result of the first calculation.

23. The apparatus of any of claim 20 wherein the estimating unit is configured to, in the estimation process, for each of the at least two sensors, calculate the state vector of a current sample (k) of the signal from the respective sensor based on the state vector of a previous sample (1<−1) of the signal, using the sensor-specific model for the respective sensor computed in the selection process.

24. The apparatus of claim 23 wherein the estimating unit is configured to use the process noise values which are obtained for the at least one state parameter of the at least two sensors for calculating the state vector.

25. The apparatus of claim 23 wherein the estimating unit for calculating the state vector, is configured to:
predict a current state vector of the current sample based on a previous state vector of the previous sample and a linear model of the sensor-specific model for the respective sensor; and
update the predicted current state vector by using a non-linear model of the sensor-specific model of the respective sensor; and
based on the one sensor computed by the selecting unit, switch the sensor-specific model to be used for estimating the biological parameter to the sensor-specific model provided for the selected one sensor.

26. The apparatus of claim 20 wherein the estimating unit is configured to preprocess the signals from each of the at least two sensors, wherein the pre-processed signals are output as the signals from the at least two sensors to the initialization process and the estimation process.
